(19) United States
(12) Patent Application Publication
(10) Pub. No.: US 2019/0323880 A1
(43) Pub. Date: Oct. 24, 2019

(54) MEASURING DEVICE

(71) Applicant: LIXIL Corporation, Tokyo (JP)

(72) Inventors: Hirokazu SHIMASAKI, Tokyo (JP); Hiroaki WATANABE, Tokyo (JP); Tsunaki ITOU, Tokyo (JP); Tomoya SASAKI, Tokyo (JP); Hideya KOIKE, Tokyo (JP)

(73) Assignee: LIXIL Corporation, Tokyo (JP)

(21) Appl. No.: 16/475,977

(22) PCT Filed: Jan. 4, 2018
(86) PCT No.: PCT/JP2018/000009

§ 371 Date: Jul. 3, 2019

(30) Foreign Application Priority Data
Jan. 6, 2017 (JP) 2017-001098

(51) Int. Cl. G01G 19/52; A47K 13/24; G01G 19/50; G01N 33/493; G01N 33/487

(52) U.S. Cl. CPC G01G 19/52; A47K 13/24; G01N 33/48707; G01N 33/493; G01G 19/50

(57) ABSTRACT

The present invention provides a measuring device which is configured to measure a body weight of a user sitting on a toilet seat, which is installed on a toilet bowl, and on which the toilet seat is installed, the device including a plurality of load measuring units that are configured to measure a load of the user acting on the toilet seat and that are disposed to protrude upward from an upper surface. The toilet seat includes a plurality of leg portions disposed to protrude downward from a lower surface, a height from the upper surface to upper ends of the load measuring units is greater than a height from the lower surface of the toilet seat to lower ends of the leg portions, and the load measuring units are disposed at different positions from the leg portions as seen in plan view.

### CROSS-REFERENCE TO RELATED APPLICATIONS

This application is a U.S. national stage application under 35 U.S.C. 371 of International Application No. PCT/JP2018/000009, filed Jan. 4, 2018, which claims priority to Japanese Application No. 2017-001098, filed Jan. 6, 2017, each of which are hereby incorporated by reference in the present disclosure in its entirety.

### FIELD OF THE INVENTION

The present disclosure relates to a measuring device.

### BACKGROUND OF THE INVENTION

A toilet apparatus with a body weight measuring function, in which a pressure sensor is installed on a lower surface of a toilet seat and which can measure the body weight of a user corresponding to a pressure value that is measured by the pressure sensor when the user sits on the toilet seat, has been proposed (refer to Patent Document 1).

Patent Document 1: Japanese Patent No. 3063245

### SUMMARY OF THE INVENTION

However, in a case where it is desired to add a body weight measuring function to a toilet apparatus in the related art, there is a problem that it is necessary to replace a toilet seat thereof with a toilet seat (dedicated toilet seat) as described in Patent Document 1 in which a pressure sensor is installed.

In addition, a difference in height such as unevenness is likely to be generated on an upper surface of a pottery toilet bowl. Therefore, in the related art, a lower surface of a toilet seat is provided with a leg portion that is capable of being elastically deformed and the leg portion is installed on an upper surface of a toilet bowl so as to even out a difference in height of the upper surface of the toilet bowl. Accordingly, when installing a pressure sensor on the toilet bowl, the toilet seat, or the like, it is desired to avoid interference between the pressure sensor and the leg portion of the toilet seat.

Embodiments of the present invention have been made in consideration of the above-described circumstances. Provided is a measuring device which is capable of being installed without interfering with a leg portion of a toilet seat installed in a toilet apparatus in the related art and which is capable of measuring a body weight.

In order to achieve the above-described object, embodiments of the present invention adopt the following means.

In some embodiments, there is provided a measuring device which is configured to measure a body weight of a user sitting on a toilet seat, which is installed on a toilet bowl, and on which the toilet seat is installed, the device including a plurality of load measuring units that are configured to measure a load of the user acting on the toilet seat and that are disposed to protrude upward from an upper surface of the measuring device. The toilet seat can include a plurality of leg portions disposed to protrude downward from a lower surface of the toilet seat, a height from the upper surface to upper ends of the load measuring units is greater than a height from the lower surface of the toilet seat to lower ends of the leg portions, and the load measuring units are disposed at different positions from the leg portions as seen in plan view.

The measuring device configured as described above includes the load measuring units that are configured to measure the load of the user acting on the toilet seat and the measuring device is installed on the toilet bowl. In addition, the toilet seat is installed on the measuring device. Accordingly, it is possible to install the measuring device on the toilet bowl and use the toilet seat without replacement of the toilet seat installed in a conventional toilet apparatus.

In some embodiments, since the plurality of load measuring units provided to protrude upward from the upper surface of the measuring device and the plurality of leg portions provided to protrude downward from the lower surface of the toilet seat are disposed at different positions as seen in plan view, the load measuring unit and the leg portions do not interfere with each other as seen in plan view.

Furthermore, since the height from the upper surface of the measuring device to the upper ends of the load measuring units is greater than the height from the lower surface of the toilet seat to the lower ends of the leg portions, the load measuring units are capable of supporting the lower surface of the toilet seat and the lower ends of the leg portions are not supported by the upper surface of the measuring device. Therefore, it is possible to accurately measure the load of the user acting on the toilet seat.

In some embodiments, in the measuring device related to the first aspect, the plurality of load measuring units may be disposed to be separated from each other in a lateral direction. The leg portions may be disposed between the load measuring units on front and rear sides and the leg portions may be disposed behind the load measuring units on the rear side.

In the case of the measuring device configured as described, the leg portions are disposed between the load measuring units on the front and rear sides and the leg portions are disposed behind the load measuring units on the rear side. Accordingly, the load measuring units on the front and rear sides support the toilet seat while being dispersed. Therefore, the toilet seat is not bent downward and it is possible to more accurately measure the load of the user.

In some embodiments, the measuring device related to the first aspect or the second aspect may further include a urine sensor that includes an electrode electrolyzing urine and a control unit that is configured to operate the urine sensor and calculate an amount of a component contained in the urine according to a result of electrolysis performed by the electrode.

The measuring device configured as described has the urine sensor that includes the electrodes electrolyzing urine and the control unit that is configured to operate the urine sensor and calculate the amount of the component contained in urine according to the result of the electrolysis performed by the electrode. Accordingly, it is possible to measure not only the body weight of the user but also the amount of a desired component in urine.

In some embodiments, the measuring device related to any one of the first to third aspects may further include a handle that is configured to measure a body fat percentage and that is configured to be gripped.

Since the measuring device configured as described above includes the handle that is configured to measure a body fat percentage and that is configured to be gripped, it is possible to measure not only the body weight of the user but also the body fat percentage.

In some embodiments, in the measuring device related to any one of the first to fourth aspects, a first connection port that is electrically connected to a toilet seat box openably and closably supporting the toilet seat may be provided, and a second connection port that is electrically connected to an external power source may be provided.

In the case of the measuring device configured as described above, the first connection port that is electrically connected to the toilet seat box is provided, and the second connection port that is electrically connected to the external power source is provided. Therefore, it is possible to supply power to the measuring device and the toilet seat box by electrically connecting a connection port provided in an existing toilet seat box and the first connection port to each other and electrically connecting the second connection port and the external power source to each other. In an aspect in which no measuring device is installed, a cable connected to the connection port provided in the existing toilet seat box is connected to the external power source. When installing the measuring device, it is possible to supply power to the measuring device and the toilet seat box by removing the cable from the external power source, connecting the cable to the first connection port of the measuring device, and connecting a cable connected to the second connection port to the external power source.

In some embodiments, it is possible to install the measuring device without interfering with leg portions of a toilet seat installed on a conventional toilet apparatus and to measure a body weight.

### BRIEF DESCRIPTION OF THE DRAWINGS

FIG. 1 is a perspective view of a flush toilet that includes a measuring device according to some embodiments of the present invention;

FIG. 2 is a side view of the measuring device according to some embodiments of the present invention;

FIG. 3 is a vertical sectional view of the measuring device according to some embodiments of the present invention cut in a lateral direction;

FIG. 4 is a perspective view of the measuring device according to some embodiments of the present invention as seen from an upper side;

FIG. 5 is a perspective view of the measuring device according to some embodiments of the present invention as seen from a lower side;

FIG. 6 is a perspective view showing a state in which an upper cover is removed from the measuring device according to some embodiments of the present invention;

FIG. 7 is a perspective view showing a state in which the upper cover is removed from the measuring device according to some embodiments of the present invention;

FIG. 8 is a perspective view of a toilet seat installed on the measuring device according to some embodiments of the present invention;

FIG. 9 is a plan view showing a disposition relationship between load measuring units of the measuring device and leg portions of the toilet seat in a flush toilet that includes the measuring device according to some embodiments of the present invention;

FIG. 10 is a side view of a measuring device according to some embodiments of the present invention; and

FIG. 11 is a side view of a measuring device according some embodiments of the present invention.

### DETAILED DESCRIPTION OF THE INVENTION

A measuring device according to embodiments of the present invention will be described.

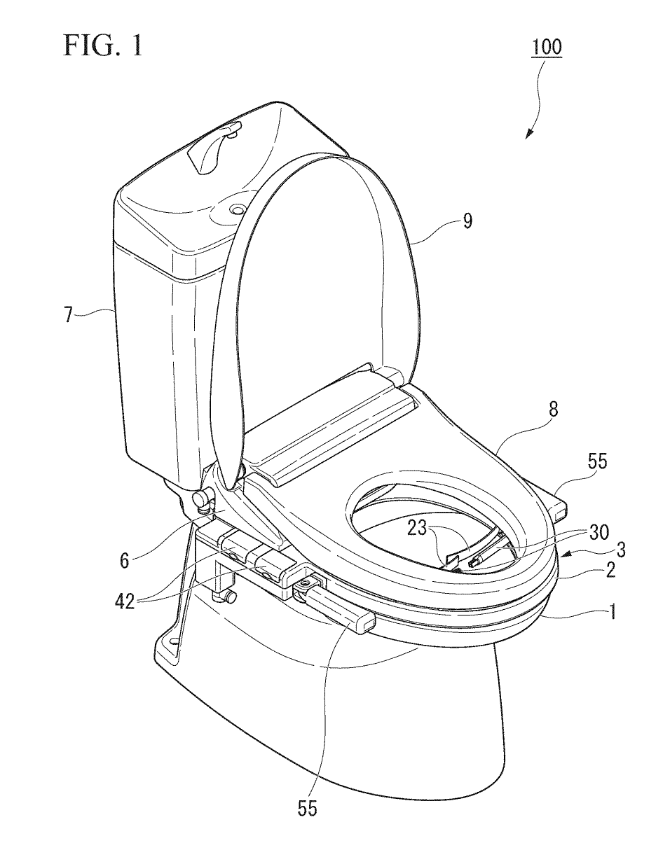

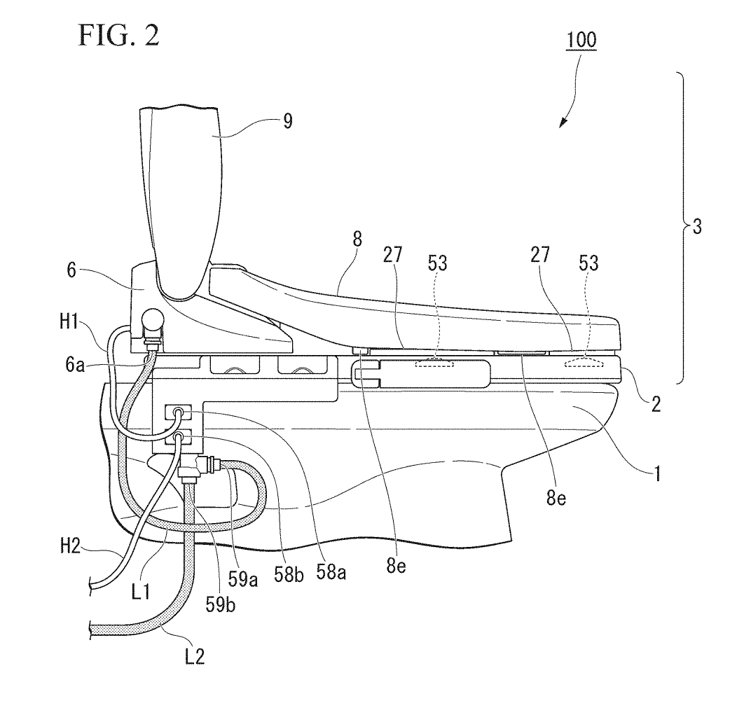

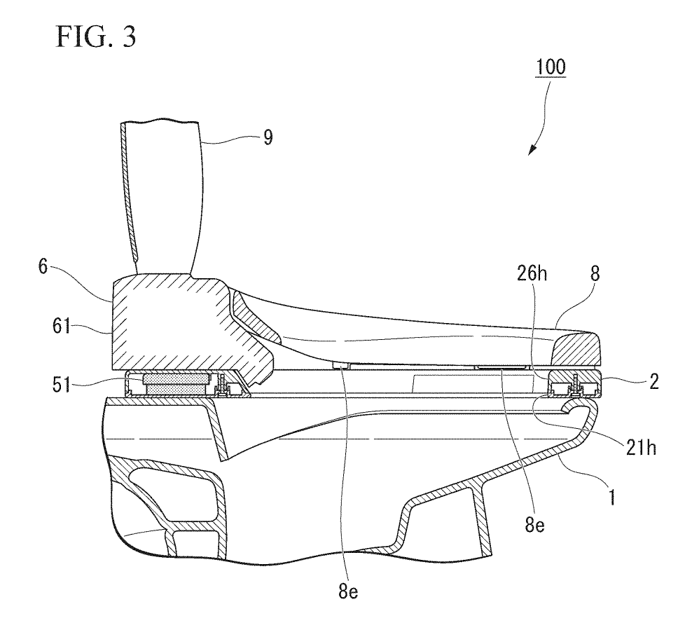

FIG. 1 is a perspective view of a flush toilet that includes the measuring device according to some embodiments of the present invention. FIG. 2 is a side view of the measuring device according to some embodiments of the present invention as seen from a right side. FIG. 3 is a vertical sectional view of the measuring device according to some embodiments of the present invention cut in a lateral direction.

As shown in FIGS. 1 to 3, a flush toilet 100 includes a toilet bowl 1, a toilet seat device 3, and a cleaning water tank 7.

The toilet seat device 3 is installed on the toilet bowl 1. The toilet seat device 3 includes a measuring device 2, a toilet seat box 6, a toilet seat 8, and a toilet lid 9.

Here, a direction which the face of a user sitting on the toilet seat 8 faces will be referred to as a frontward direction and a direction which the back of the user sitting on the toilet seat 8 faces will be referred to as a rearward direction. In addition, a width direction of the flush toilet 100, which is a horizontal direction orthogonal to the lateral direction, will be referred to as a lateral direction. In addition, a left side and a right side may be referred to as lateral sides.

The measuring device 2 is interposed between the toilet bowl 1 and the toilet seat 8. In other words, the measuring device 2 is fixed onto an upper surface of the toilet bowl 1 and the toilet seat 8 is disposed on the measuring device 2.

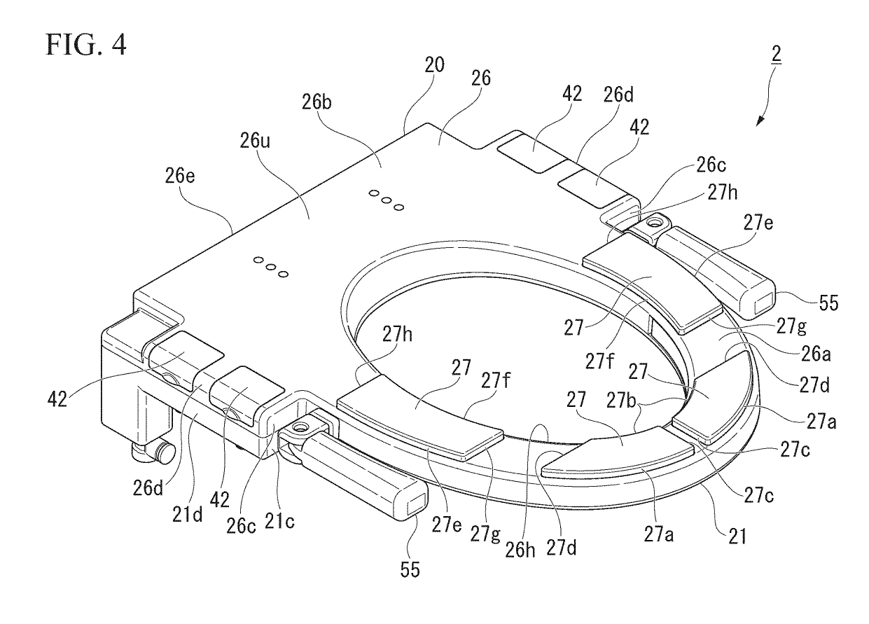

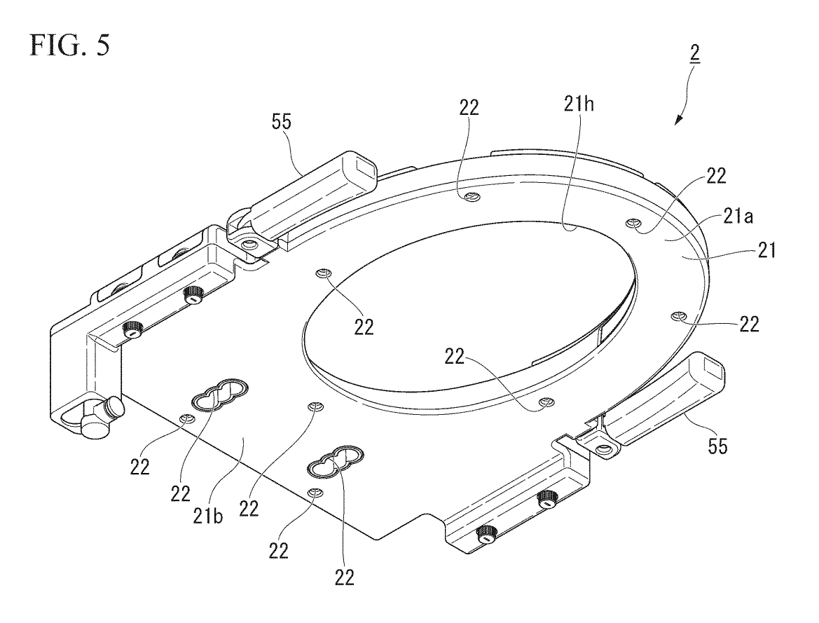

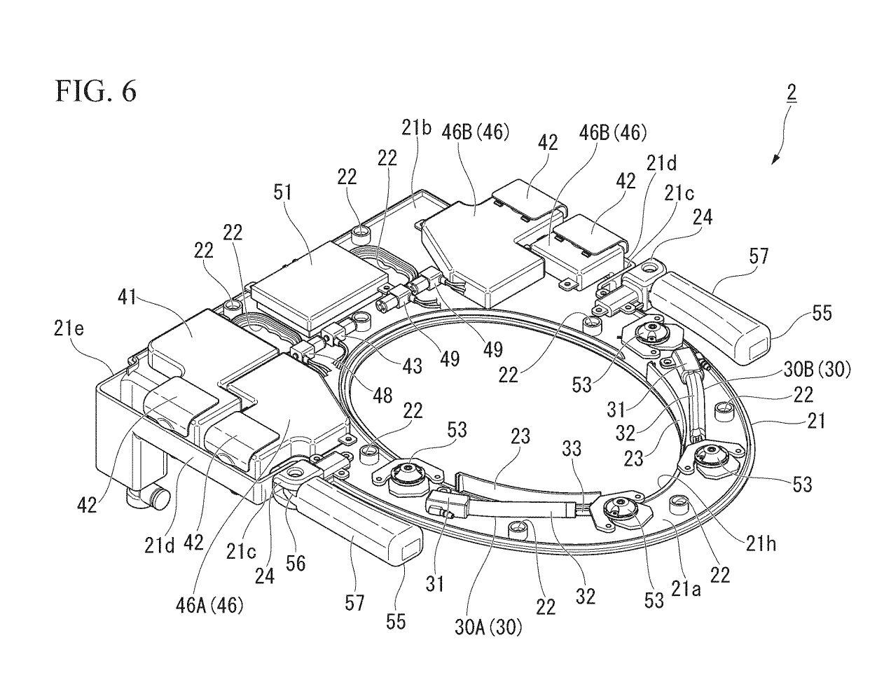

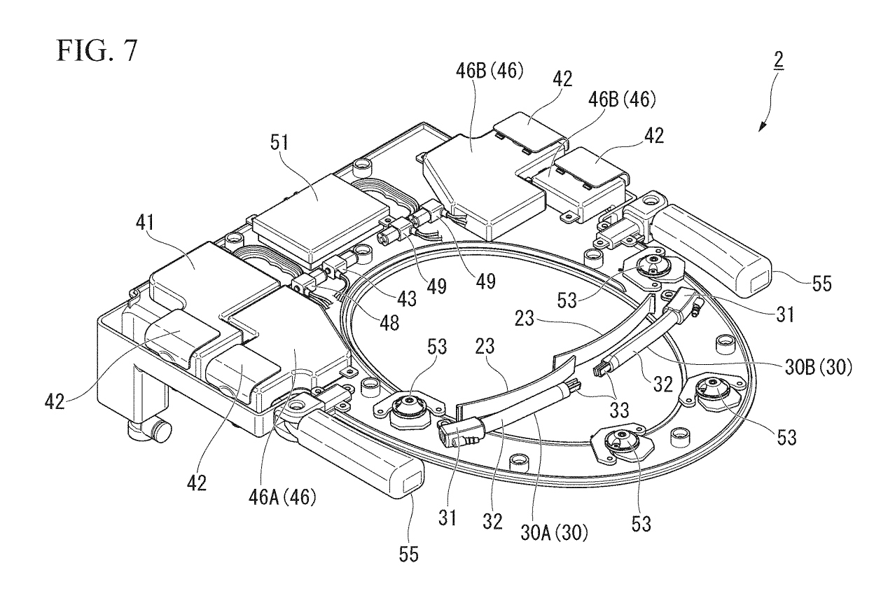

FIG. 4 is a perspective view of the measuring device 2 as seen from an upper side. FIG. 5 is a perspective view of the measuring device 2 as seen from a lower side. FIG. 6 is a perspective view showing a state, in which an upper cover 26 is removed from the measuring device 2 and urine sensors 30 are disposed at accommodation positions, as seen from the upper side. FIG. 7 is a perspective view showing a state, in which the upper cover 26 is removed from the measuring device 2 and the urine sensors 30 are disposed at measurement positions, as seen from the upper side.

As shown in FIGS. 4 to 7, the measuring device 2 includes a casing 20 that is approximately O-shaped as seen in plan view, the urine sensors 30 that are disposed inside the casing 20, a preservative solution tank 41, calibration solution tanks 46, a urine measurement control unit (control unit) 51, load cells (load measuring units) 53, and body fat measuring bodies (handles) 55.

The casing 20 includes a lower cover 21 that is fixed to the toilet bowl 1 and the upper cover 26 that is provided above the lower cover 21. Each of the lower cover 21 and the upper cover 26 includes an opening 20h that is approximately oval as seen in plan view.

As shown in FIGS. 5 and 6, the lower cover 21 includes an annular portion 21a that is formed in an annular shape as seen in plan view and an extending portion 21b that extends in both of a rightward direction and a leftward direction and in the rearward direction at a rear portion of the annular portion 21a.

The annular portion 21a has the approximately same shape as the toilet seat 8 as seen in plan view and is formed in accordance with the shape of the toilet seat 8. The extending portion 21b is formed to have a shape that extends laterally outward further than the toilet seat 8 as seen in plan view.

An outer edge of the extending portion 21b is formed by front wall portions 21c that extend in both of the rightward direction and the leftward direction from the rear portion of the annular portion 21a, side wall portions 21d that extend rearward from end portions of the front wall portions 21c, and a rear wall portion 21e that connects rear end portions of a pair of the side wall portions 21d to each other and that extends in the lateral direction. The front wall portions 21c, the side wall portions 21d, and the rear wall portion 21e rise upward from a lower surface of the extending portion 21b. An opening 21h that is approximately oval as seen in plan view is formed inward of the annular portion 21a.

A plurality of attachment holes 22 that penetrate the lower cover 21 in a vertical direction are formed in the lower cover 21. Screws or attachment tools which are not shown are inserted into the attachment holes 22 and are screwed into the toilet bowl 1.

As shown in FIG. 4, the upper cover 26 is formed to have the approximately same shape as the lower cover 21 as seen in plan view. The upper cover 26 includes an annular portion 26a that is formed in an annular shape as seen in plan view and an extending portion 26b that extends in both of the rightward direction and the leftward direction and in the rearward direction at a rear portion of the annular portion 26a.

An outer edge of the extending portion 26b is formed by front wall portions 26c that extend in both of the rightward direction and the leftward direction from the rear portion of the annular portion 26a, side wall portions 26d that extend rearward from end portions of the front wall portions 26c, and a rear wall portion 26e that connects rear end portions of a pair of the side wall portions 26d to each other and that extends in the lateral direction. The front wall portions 26c, the side wall portions 26d, and the rear wall portion 26e are bent downward from an upper surface 26u of the extending portion 26b. An opening 26h that is approximately oval as seen in plan view is provided inward of the annular portion 26a. The opening 26h of the upper cover 26 communicates with the opening 21h of the lower cover 21.

Lower ends of the front wall portions 21c of the lower cover 21 abut onto upper ends of the front wall portions 26c of the upper cover 26, lower ends of the side wall portions 21d of the lower cover 21 abut onto upper ends of the side wall portions 26d of the upper cover 26, and a lower end of the rear wall portion 21e of the lower cover 21 abuts onto an upper end of the rear wall portion 26e of the upper cover 26.

As shown in FIG. 6, the urine sensors 30, the preservative solution tank 41, the calibration solution tanks 46, the urine measurement control unit 51, and the load cells 53 are provided on an upper surface of the lower cover 21. The upper cover 26 is provided so as to cover the urine sensors 30, the preservative solution tank 41, the calibration solution tank 46, the urine measurement control unit 51 and the load cells 53. In other words, the urine sensors 30, the preservative solution tank 41, the calibration solution tanks 46, the urine measurement control unit 51, and the load cells 53 are accommodated between the lower cover 21 and the upper cover 26. As shown in FIG. 4, the upper surface 26u of the upper cover 26 is formed as a flat surface.

As shown in FIG. 6, the urine sensors 30 are provided on both of right and left sides of the annular portion 21a of the lower cover 21. Each urine sensor 30 includes a base portion 31 that is rotatably fixed to the lower cover 21, an arm portion 32 that extends forward from the base portion 31 and is formed in a hollow shape, and a urine measuring unit 33 that is disposed inside the arm portion 32.

A tip end of the urine measuring unit 33 protrudes further than a tip end of the arm portion 32. The urine measuring unit 33 includes an electrode (not shown) that is capable of electrolyzing urine.

The urine sensor covers 23 are provided at the edge of the opening 21h of the lower cover 21 so as to correspond to positions at which the urine sensors 30 are disposed.

The urine sensors 30 and the urine sensor covers 23 are connected to a drive motor (not shown).

The urine sensors 30 and the urine sensor covers 23 swing around the base portions 31 from positions (accommodation positions) at which the urine sensors 30 and the urine sensor covers 23 are disposed on the upper surface of the lower cover 21 as shown in FIG. 6 to positions (measurement positions) at which tip end sides thereof protrude toward the opening 21h of the lower cover 21 as shown in FIG. 7 by driving the drive motor based on a signal from the urine measurement control unit 51. As seen in plan view (as seen in direction in which measuring device 2 and toilet seat 8 are arranged), the urine sensors 30 disposed at the accommodation positions (refer to FIG. 6) are disposed to overlap the toilet seat 8.

As shown in FIG. 6, in some embodiments, a urine sensor (urine sugar measuring sensor) 30A on a right side from among a pair of the urine sensors 30 is capable of measuring urine sugar and a urine sensor (urine salinity measuring sensor) 30B on a left side is capable of measuring urine salinity.

The preservative solution tank 41 is provided on a right side of the lower cover 21. In the preservative solution tank 41, a preservative solution (liquid) for stabilizing the electric current or voltage of the urine sugar measuring sensor 30A is stored. The preservative solution tank 41 is connected to a pump 43 via a tube (not shown). The pump 43 is connected to the urine sugar measuring sensor 30A via a tube (not shown). The pump 43 is driven according to a signal from the urine measurement control unit 51 and the preservative solution in the preservative solution tank 41 flows through the tube and the preservative solution is supplied to the urine sugar measuring sensor 30A. Accordingly, a tip end portion of the urine sugar measuring sensor 30A is immersed in the preservative solution inside a container (not shown).

The calibration solution tanks 46 are disposed on both of right and left sides of the lower cover 21. In a calibration solution tank 46A on the right side, a calibration solution for calibration of the urine sugar measuring sensor 30A is stored. In calibration solution tanks 46B on the left side, a calibration solution for calibration of the urine salinity measuring sensor 30B is stored. The calibration solution tank 46A is connected to a pump 48 via a tube (not shown). The pump 48 is connected to the urine sugar measuring sensor 30A via a tube (not shown). The pump 48 is driven according to a signal from the urine measurement control unit 51, and the calibration solution in the calibration solution tank 46A flows through the tube and is supplied to the urine sugar measuring sensor 30A at a predetermined frequency. Accordingly, the calibration solution drips onto the urine sugar measuring sensor 30A inside a container (not shown). In addition, the calibration solution tanks 46B are connected to pumps 49 via a tube (not shown). The pumps 49 are connected to the urine salinity measuring sensor 30B via a tube (not shown). The pumps 49 are driven according to a signal from the urine measurement control unit 51, and the calibration solution in the calibration solution tanks 46B flows through the tube and is supplied to the urine salinity measuring sensor 30B at a predetermined frequency. Accordingly, the calibration solution drips onto the urine salinity measuring sensor 30B inside a container (not shown).

Each of the preservative solution tank 41 and the calibration solution tanks 46 has a lid 42 that is capable of being opened and closed. Specifically, as shown in FIG. 1, as seen in plan view, the lids 42 are disposed to protrude from the lateral side of the toilet seat 8 (laterally outward). In other words, as shown in FIG. 6, the lids 42 are disposed behind the front wall portions 21c of the lower cover 21. As shown in FIG. 4, each lid 42 is disposed along the upper surface of the extending portion 26b of the upper cover 26 of the casing 20 installed below the toilet seat 8. Therefore, a user is capable of opening and closing the lids 42 and is capable of supplying the preservative solution or the calibration solution without removing the toilet seat 8 (with toilet seat 8 installed).

The urine measurement control unit 51 is provided on a rear portion of the lower cover 21. Specifically, as seen in plan view, the urine measurement control unit 51 is disposed ahead of the rear wall portion 21e in the extending portion 21b of the lower cover 21 and is disposed along the rear wall portion 21e. As shown in FIGS. 1 and 3, as seen in plan view (as seen in direction in which measuring device 2 and toilet seat 8 are arranged), the urine measurement control unit 51 is disposed to overlap the toilet seat 8. In addition, the urine measurement control unit 51 may measure a body weight according to the result of measurement performed by the load cells 53 and may measure a body fat percentage according to the result of measurement performed by the body fat measuring bodies 55.

As shown in FIG. 6, the urine measurement control unit 51 outputs a signal for driving the pumps 43, 48 and 49, outputs a signal to the drive motor that causes the urine sensors 30 to swing between the accommodation positions and the measurement positions, or calculates the amount of components according to the result of electrolysis in the urine sensors 30. In addition, the urine measurement control unit 51 may have a function of transmitting a signal for outputting a measurement value measured by the urine sensors 30 to a display unit (not shown).

The load cells 53 measure the loads of the user acting on the toilet seat 8. A plurality of the load cells 53 are provided on the lower cover 21 and the load cells 53 are electrically connected to the toilet seat box 6.

In some embodiments, four load cells 53 are disposed in a bilaterally symmetrical manner and are disposed to be separated from each other in the lateral direction. As seen in plan view, the load cells 53 are disposed to overlap the toilet seat 8.

Two load cells 53 on a front side are disposed to be separated from each other in the lateral direction near tip end sides (front end sides) of the openings 21h and 26h of the toilet seat 8. Front portions of the thighs of the user are placed above the load cells 53. Two load cells 53 on a rear side are disposed on the right and left sides of the toilet seat 8 near the centers of the openings 21h and 26h in the lateral direction. Rear portions of the thighs of the user are placed above the load cells 53.

As shown in FIG. 4, load cell covers (load measuring units) 27 are provided on the upper cover 26 at positions corresponding to the load cells 53. That is, two load cell covers 27 on the front side are disposed to be separated from each other in the lateral direction at the toilet seat 8 near the tip end sides (front end sides) of the openings 21h and 26h. The front portions of the thighs of the user are placed above the load cell covers 27. Two load cell covers 27 on the rear side are disposed on the right and left sides of the toilet seat 8 near the centers of the openings 21h and 26h in the lateral direction. The rear portions of the thighs of the user are placed above the load cell covers 27.

The load cell covers 27 are disposed to cover the load cells 53 and protrude upward from the upper surface 26u of the upper cover 26.

Each of the load cell covers 27 on the front side includes an outer curved portion 27a that is formed to be curved along an outer edge of the toilet seat 8, an inner curved portion 27b that is formed to be curved along the openings 21h and 26h of the toilet seat 8, an inner side wall portion 27c that connects the outer curved portion 27a and the inner curved portion 27b to each other and extends in an approximately lateral direction, and an outer side wall portion 27d that connects the outer curved portion 27a and the inner curved portion 27b to each other and extends in an approximately lateral direction. As seen in plan view, each of the load cell covers 27 on the front side is formed to have a long shape extending in the lateral direction.

Each of the load cell covers 27 on the rear side includes an outer curved portion 27e that is formed to be curved along the outer edge of the toilet seat 8, an inner curved portion 27f that is formed to be curved along the openings 21h and 26h of the toilet seat 8, an inner side wall portion 27g that connects a front end portion of the outer curved portion 27e and a front end portion of the inner curved portion 27f to each other and extends in an approximately lateral direction, and an outer side wall portion 27h that connects a rear end portion of the outer curved portion 27e and a rear end portion of the inner curved portion 27f to each other and extends in an approximately lateral direction. As seen in plan view, each of the load cell covers 27 on the rear side is formed to have a long shape extending in the lateral direction.

The load cells 53 output measured loads to the toilet seat box 6 in the form of load signals. That is, loads that act on the load cells 53 when the user sits on the toilet seat 8 are converted into electric signals by the load cells 53 and are transmitted to the toilet seat box 6 in the form of the load signals.

The body fat measuring bodies 55 are provided on the right and left sides of the casing 20. Specifically, the annular portion 21a of the lower cover 21 includes attachment portions 24 that are provided ahead of the front wall portions 21c and laterally inward of the front wall portions 21c. Each of the body fat measuring bodies 55 includes a base portion 56 that is rotatably fixed to the attachment portion 24 provided in the lower cover 21 and a grip portion 57 that extends from the base portion 56 and that is capable of being gripped. The body fat measuring bodies 55 cause a minute amount of electric current to flow toward the body with the user gripping the grip portions 57 and measure the resistance by using an impedance method or the like to measure the body fat percentage or the like. The grip portions 57 are capable of swinging from positions (accommodation positions) at which tip ends thereof face the front side as shown in FIG. 6 to positions (measurement positions) at which the tip ends thereof face lateral sides by rotating 90° within a plane.

As shown in FIG. 2, the connection ports 58a and 58b are provided in the measuring device 2. A cable H1 connected to the connection port (first connection port) 58a is connected to a connection port of the toilet seat box 6. A cable H2 connected to the connection port (second connection port) 58b is connected to an external power source (not shown). The connection ports 58a and 58b are preferably provided on a rear side or a lateral side of the measuring device.

The water supply ports 59a and 59b are provided in the measuring device 2. A pipe L1 connected to the water supply port 59a is connected to a water supply port 6a of the toilet seat box 6. A pipe L2 connected to the water supply port 59b is connected to an external water supply pipe (not shown). The water supply ports 59a and 59b are preferably provided on the rear side or the lateral side of the measuring device. Water that is introduced into the measuring device 2 via the water supply port 59b and the pipe L2 from the external water supply pipe is used for the cleaning or the like of the urine sensors 30. In addition, water that is introduced into the toilet seat box 6 via the water supply port 6a and the pipe L1 from the water supply port 59a of the measuring device 2 is used for private part cleaning or the like.

As shown in FIG. 3, the toilet seat box 6 is installed on the measuring device 2 at a rear portion of the measuring device 2. The toilet seat box 6 includes a casing 61, various functional components (not shown) such as a toilet seat opening and closing mechanism that is disposed inside the casing 61 and opens and closes the toilet seat 8, a toilet lid opening and closing mechanism that opens and closes the toilet lid 9, and a private part cleaning device (cleaning function unit) that cleans private parts of a human body, and a main body control unit (not shown) that controls the various functional components.

As shown in FIG. 1, the cleaning water tank 7 is fixed onto the toilet bowl 1 at a position behind the toilet seat box 6. In the cleaning water tank 7, cleaning water for cleaning the toilet bowl 1 is stored.

The toilet seat 8 is formed to be approximately 0-shaped as seen in plan view. A rear portion thereof is pivotally supported by the toilet seat box 6 and the toilet seat 8 is capable of swinging relative to the toilet bowl 1 between an erected state and a lying state. The toilet seat 8 is installed on the measuring device 2 in the lying state (refer to FIG. 1).

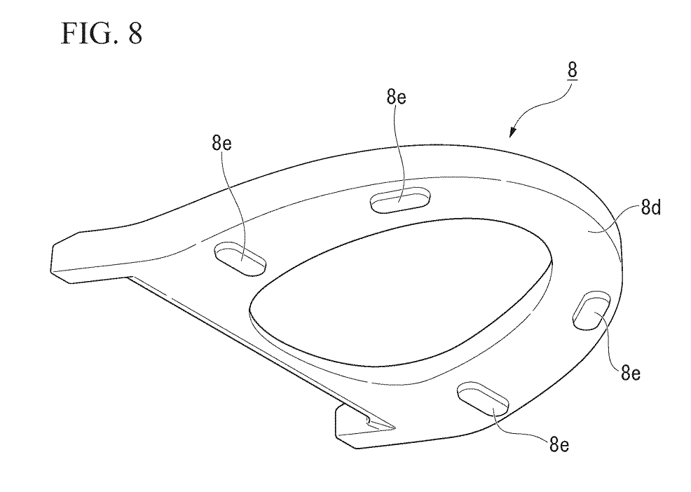

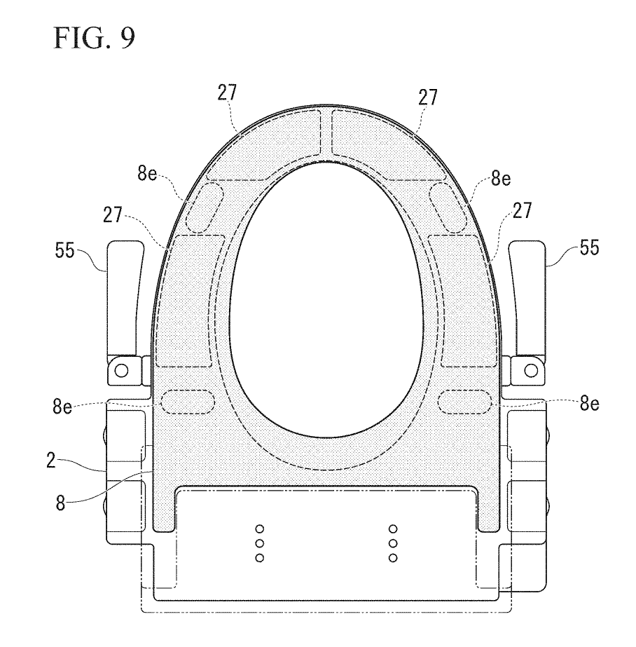

FIG. 8 is a perspective view of the toilet seat 8 as seen from the lower side. FIG. 9 is plan view showing a disposition relationship between the load cell covers 27 of the measuring device 2 and leg portions 8e of the toilet seat 8 in the flush toilet 100.

As shown in FIG. 8, a plurality of the leg portions 8e are provided on a lower surface 8d of the toilet seat 8. In some embodiments, four leg portions 8e are disposed in a bilaterally symmetrical manner and are disposed to be separated from each other in the lateral direction.

As shown in FIG. 9, the load cell covers 27 and the leg portions 8e are disposed at different positions from each other as seen in plan view. In other words, the leg portions 8e on the front side are disposed between the load cell covers 27 on the front side and the load cell covers 27 on the rear side. The leg portions 8e on the rear side are disposed behind the load cell covers 27 on the rear side.

In addition, as seen in a side view shown in FIG. 2, a height from the upper surface 26u of the upper cover 26 of the measuring device 2 to the upper ends of the load cell covers 27 is greater than a height from the lower surface 8d of the toilet seat 8 to lower ends of the leg portions 8e. In other words, a height from the upper surface 26u of the upper cover 26 of the measuring device 2 to the upper ends of the load cell covers 27 in a direction in which the measuring device 2 and the toilet seat 8 are arranged is greater than a height from the lower surface 8d of the toilet seat 8 to lower ends of the leg portions 8e in a direction in which the measuring device 2 and the toilet seat 8 are arranged. Accordingly, the load cell covers 27 is capable of supporting the lower surface 8d of the toilet seat 8 and the lower ends of the leg portions 8e of the toilet seat 8 are not supported by the upper surface 26u of the upper cover 26 of the measuring device 2.

The toilet lid 9 is pivotally supported by the toilet seat box 6 and is capable of swinging relative to the toilet bowl 1 between an erected state and a lying state.

Next, an operation of the measuring device 2 in the flush toilet 100 configured as described above will be described.

When the user sits on the toilet seat 8, a sitting sensor (not shown) detects that the user has sat thereon. The urine measurement control unit 51 transmits a measurement position movement signal to the drive motor. Accordingly, the drive motor causes the urine sugar measuring sensor 30A, the urine salinity measuring sensor 30B, and the urine sensor covers 23 to move to the measurement positions from the accommodation positions. The grip portions 57 of the body fat measuring bodies 55 may be configured to move to the measurement positions from the accommodation positions according to the signal from the urine measurement control unit 51 at this time.

When the user urinates, each of the electrodes of the urine sugar measuring sensor 30A and the urine salinity measuring sensor 30B performs electrolysis. The urine measurement control unit 51 calculates urine sugar and urine salinity according to the result of the electrolysis.

Meanwhile, when the user sits on the toilet seat 8, loads act on the load cells 53. The loads are converted into electric signals by the load cells 53 and are transmitted to the urine measurement control unit 51. The urine measurement control unit 51 adds up the loads indicated by the electric signals to calculate the body weight of the user.

In addition, when the user grips the grip portions 57 of the body fat measuring bodies 55, the urine measurement control unit 51 causes a minute amount of electric current to flow toward the body and measures the resistance by using an impedance method or the like to measure the body fat percentage or the like.

The urine measurement control unit 51 may cause the result of the measurement to be displayed on a display unit (not shown).

In the case of the flush toilet 100 configured as described above, the measuring device 2, which includes the load cells 53 that are configured to measure the loads of the user acting on the toilet seat 8, is installed on the toilet bowl 1. In addition, the toilet seat 8 is installed on the measuring device 2. Accordingly, it is possible to install the measuring device 2 on the toilet bowl 1 and use the toilet seat 8 as it is without replacement of the toilet seat 8 installed on the conventional flush toilet 100.

In addition, since the plurality of load cells 53 provided to protrude upward from the upper surface 26u of the upper cover 26 of the measuring device 2 and the plurality of leg portions 8e provided to protrude downward from the lower surface 8d of the toilet seat 8 are disposed at different positions as seen in plan view (as seen in direction in which measuring device 2 and toilet seat 8 are arranged), the load cells 53 and the leg portions 8e do not interfere with each other as seen in plan view.

Furthermore, since a height from the upper surface 26u of the upper cover 26 of the measuring device 2 to the upper ends of the load cells 53 is greater than a height from the lower surface 8d of the toilet seat 8 to the lower ends of the leg portions 8e, the load cells 53 is capable of supporting the lower surface 8d of the toilet seat 8 and the lower ends of the leg portions 8e of the toilet seat 8 are not supported by the upper surface 26u of the upper cover 26 of the measuring device 2. Therefore, it is possible to accurately measure the loads of the user acting on the toilet seat 8.

In addition, the leg portions 8e are disposed between the load cells 53 on the front and rear sides and behind the load cells 53 on the rear side. Accordingly, the load cells 53 on the front and rear sides support the toilet seat 8 while being dispersed. Therefore, the toilet seat 8 is not bent downward and it is possible to more accurately measure the loads of the user.

In addition, the measuring device 2 includes the urine sensors 30 that include the electrodes capable of electrolyzing urine and the urine measurement control unit 51 that operates the urine sensors 30 and calculates the amount of components contained in urine according to the result of the electrolysis performed by the electrodes. Accordingly, it is possible to measure not only the body weight of the user but also the amount of a desired component in urine.

In addition, since the measuring device 2 includes the body fat measuring bodies 55 that are capable of measuring a body fat percentage and that are capable of being gripped, it is possible to measure not only the body weight of the user but also the body fat percentage.

In addition, the connection port 58a to which the cable H1 connected to the connection port of the toilet seat box 6 is connected and the connection port 58b to which the cable H2 connected to the external power source is connected are provided in the measuring device 2. Therefore, it is possible to supply power to the measuring device 2 and the toilet seat box 6 by electrically connecting a connection port provided in an existing toilet seat box and the connection port 58a to each other and electrically connecting the connection port 58b and the external power source to each other. In an aspect in which no measuring device 2 is installed, the cable H1 connected to the connection port provided in the existing toilet seat box 6 is connected to the external power source. When installing the measuring device 2, it is possible to supply power to the measuring device 2 and the toilet seat box 6 by removing the cable H1 from the external power source, connecting the cable H1 to the connection port 58a of the measuring device 2, and connecting the cable H2 connected to the connection port 58b to the external power source.

In addition, since the load cells 53 are covered by the load cell covers 27, the load cells 53 are not likely to be contaminated.

Note that, embodiments of the present invention are not limited to the measuring device according to the present invention and the measuring device 2 according to the above-described embodiments and appropriate modifications, substitutions, and the like can be made without departing from the scope of the present invention.

Hereinafter, examples of embodiments of the present invention will be described. The same or similar components or members as those described in the above-described embodiments will be described using the same reference numerals.

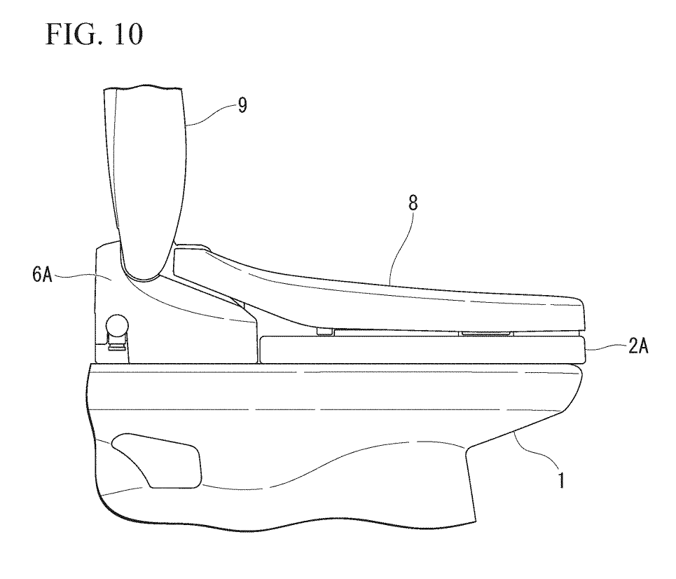

Next, a measuring device according to embodiments of the present invention will be described mainly according to FIG. 10.

FIG. 10 is a side view of the measuring device according to embodiments of the present invention as seen from a right side.

In some embodiments, the toilet seat box 6 is installed on the measuring device 2 at a rear portion of the measuring device 2. However, as shown FIG. 10, a toilet seat box 6A is positioned behind a measuring device 2A and is installed on the toilet bowl 1.

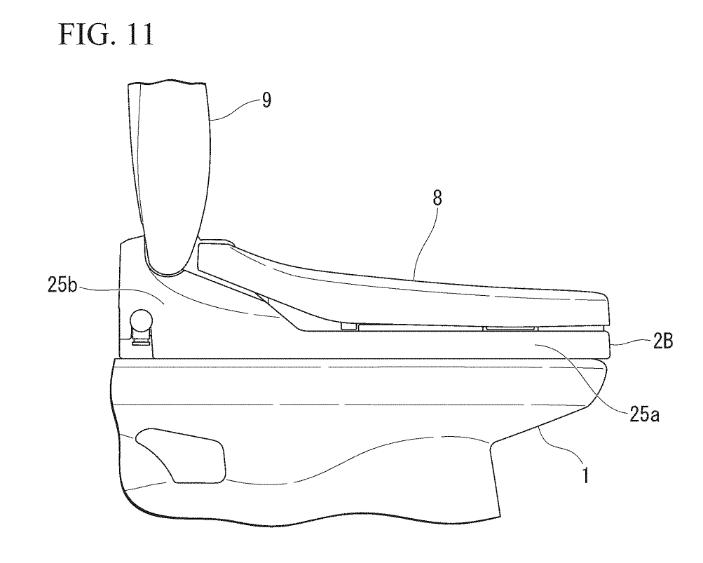

Next, a measuring device according to embodiments of the present invention will be described mainly according to FIG. 11.

FIG. 11 is a side view of the measuring device according to some embodiments of the present invention as seen from a right side.

As shown in FIG. 11, a measuring unit 2B has a configuration as the toilet seat box and a configuration as the measuring device. In other words, the toilet seat box and the measuring device are integrally formed with each other. The measuring unit 2B includes an annular portion 25a formed to be annular as seen in plan view and a rear portion body 25b that is provided to be aligned with a rear portion of the annular portion 25a and of which the height is greater than that of the annular portion 25a. In the rear portion body 25b, a tank, a urine measurement control unit, various functional components, a main body control unit, and the like are disposed.

Note that, the assembling procedure described in the above-described embodiments, the shapes, combinations, and the like of the constituent members are merely examples, and various modifications can be made according to design requirements and the like without departing from the scope of the present invention.

In addition, it is possible to replace components in the above-described embodiments with known components without departing from the spirit of the present invention.

In addition, embodiments of the present invention is not limited by the above description and is limited only by the appended claims.

According to the measuring device in embodiments of the present invention, it is possible to install the measuring device without interfering with leg portions of a toilet seat installed on a toilet apparatus in the related art and to measure a body weight.

1. A measuring device for measuring a body weight of a user sitting on a toilet seat, the measuring device comprising:
an upper cover;
a plurality of load measuring units configured to measure a load of a user sitting on a toilet seat and disposed protruding upward from an upper surface of an upper cover of the measuring device,
wherein a height of each of the load measuring units of the plurality of load measuring units measured from the upper surface of the upper cover is greater than a height of each leg portion of a plurality of leg portions of a toilet seat measured from a lower surface of the toilet seat, the plurality of leg portions disposed protruding downward from the lower surface of the toilet seat, and
wherein, when the measuring device is installed on a toilet bowl, the load measuring units of the measuring device are disposed at non-overlapping positions from the plurality of leg portions of the toilet seat when the toilet seat is installed on the measuring device.

2. The measuring device of claim 1,
wherein each load measuring unit of the plurality of load measuring units is separate in a lateral direction, and
wherein, when the toilet seat is installed on the measuring device, a first and a second leg portion of the plurality of leg portions are disposed behind the plurality of load measuring units on a rear side of the toilet seat and a third and a fourth leg portion of the plurality of leg portions are each disposed towards a front side of the toilet seat, each between two load measuring units of the plurality of load measuring units.

3. The measuring device of claim 1, comprising:
a urine sensor that includes an electrode electrolyzing urine; and
a control unit configured to operate the urine sensor and calculate an amount of a component contained in the urine according to a result of electrolysis performed by the electrode.

4. The measuring device of claim 1, comprising:
a handle configured to measure a body fat percentage when gripped by a user.

5. The measuring device of claim 1, comprising:
a first connection port and a second connection port, the first connection port being electrically connected to a toilet seat box openably and closably supporting the toilet seat, the second connection port being electrically connected to an external power source.

6. The measuring device of claim 2, comprising:
a urine sensor that includes an electrode electrolyzing urine; and
a control unit configured to operate the urine sensor and calculate an amount of a component contained in the urine according to a result of electrolysis performed by the electrode.

7. The measuring device of claim 2, comprising:
a handle configured to measure a body fat percentage when gripped by a user.

8. The measuring device of claim 3, comprising:
a handle configured to measure a body fat percentage when gripped by a user.

9. The measuring device of claim 6, comprising:
a handle configured to measure a body fat percentage when gripped by a user.

10. The measuring device of claim 2, comprising:
a first connection port and a second connection port, the first connection port being electrically connected to a toilet seat box openably and closably supporting the toilet seat, the second connection port being electrically connected to an external power source.

11. The measuring device of claim 3, comprising:
a first connection port and a second connection port, the first connection port being electrically connected to a toilet seat box openably and closably supporting the toilet seat, the second connection port being electrically connected to an external power source.

12. The measuring device of claim 4, comprising:
a first connection port and a second connection port, the first connection port being electrically connected to a toilet seat box openably and closably supporting the toilet seat, the second connection port being electrically connected to an external power source.

13. The measuring device of claim 6, comprising:
a first connection port and a second connection port, the first connection port being electrically connected to a toilet seat box openably and closably supporting the toilet seat, the second connection port being electrically connected to an external power source.

14. The measuring device of claim 7, comprising:
a first connection port and a second connection port, the first connection port being electrically connected to a toilet seat box openably and closably supporting the toilet seat, the second connection port being electrically connected to an external power source.

15. The measuring device of claim 8, comprising:
a first connection port and a second connection port, the first connection port being electrically connected to a toilet seat box openably and closably supporting the toilet seat, the second connection port being electrically connected to an external power source.

16. The measuring device of claim 9, comprising:
a first connection port and a second connection port, the first connection port being electrically connected to a toilet seat box openably and closably supporting the toilet seat, the second connection port being electrically connected to an external power source.
